(19) United States
(12) Patent Application Publication
(10) Pub. No.: US 2006/0003462 A1
(43) Pub. Date: Jan. 5, 2006

(54) Dual-slot biological test meter test procedure

(76) Inventors: Qiong Wang, Shanghai (CN); Hsing Ouyang, JangHe City (TW); Ying Ouyang, JangHe City (TW); Yao Ouyang, JangHe City (TW)

Correspondence Address: PRO-TECHTOR INTERNATIONAL, 20775 Norada Court, Saratoga, CA, 95070-3018, US

(21) Appl. No.: 11/031,619

(22) Filed: Jan. 6, 2005

(30) Foreign Application Priority Data
Jul. 2, 2004 (TW) 093119994

(51) Int. Cl. G01N 33/00

(52) U.S. Cl. 436/149; 422/68.1

(57) ABSTRACT

A dual-slot biological test meter test procedure includes the steps of 1) inserting a code card into the first test slot of a dual-slot biological test meter to input predetermined parameters and then inserting a test strip into the second test slot to initiate the dual-slot biological test meter, 2) entering a first state to electrically connect the two electrodes of the inserted test it is over the threshold after application of blood sample to the reaction zone of the inserted test strip, and then entering a second stage to output a voltage to the electrodes of the inserted test strip for a predetermined length of time set by the code card; 3) entering a third stage to read the current value subject to the setting of the code card when the predetermined length of time is up, and then to calculate the test result through a computing method set by the code card subject to the current value obtained, and then to show the test result on a display.

### BACKGROUND OF THE INVENTION

1. Field of the Invention

The present invention relates generally to the test procedure of a biological test meter and more particularly, to a dual-slot biological test meter test procedure, which uses a code card to input parameters into the dual-slot biological test meter so that the dual-slot biological test meter can be used with different test strips from different suppliers.

2. Description of the Related Art

Various biological test meters have been developed for examining the concentration of glucose, cholesterol, and globubin in blood. During examination, the test strip is inserted into the meter, and then a drop of blood sample is dropped onto the reaction zone of the inserted test strip, and then the two electrodes at the reaction zone are electrically connected. After connection of electric current to the electrodes, the meter analyzes input signal, and therefore the concentration of glucose, cholesterol, and globubin in blood is obtained. However, different reagent suppliers are continuously developing different test strips. Regular biological test meters cannot fit all different test strips from different suppliers. One specific model of biological test meter may be used with specific test strips from one particular supplier. Further conventional biological test meters do not have the function of self-calibration. If a biological test meter is not well calibrated, the user cannot obtain the test result accurately.

### SUMMARY OF THE INVENTION

The present invention has been accomplished under the circumstances in view. It is the main object of the present invention to provide a dual-slot biological test meter test procedure, which enables a dual-slot biological test meter to use different test strips from different suppliers for measuring different test items. It is another object of the present invention to provide a dual-slot biological test meter test procedure, which provides a calibrate card for insertion into the dual-slot biological test meter to calibrate the linearity of the dual-slot biological test meter. It is still another object of the present invention to provide a dual-slot biological test meter test procedure, which automatically calculate the validity of the inserted test strip, refusing the use of an expired test strip. It is still another object of the present invention to provide a dual-slot biological test meter test procedure, which automatically judges the type of the inserted test strip. To achieve these and other objects of the present invention, the dual-slot biological test meter test procedure comprises the steps of: a) inserting a code card into a first test slot of a dual-slot biological test meter to input predetermined parameters into the dual-slot biological test meter and then inserting a test strip into a second test slot of the dual-slot biological test meter to initiate the dual-slot biological test meter, b) judging if the result is over the threshold or not after application of the blood sample to the reaction zone of the inserted test strip, and then entering a first state to electrically connect the two electrodes of the inserted test, strip if the result is over the threshold, and then entering a second stage to output a voltage to the electrodes of the inserted test strip for a predetermined length of time set by the code card; c) entering a third stage to read the current value subject to the setting of the code card when the predetermined length of time is up, and then to calculate the test result through a computing method set by the code card subject to the current value obtained, and then to show the test result on a display.

### BRIEF DESCRIPTION OF THE DRAWINGS

FIG. 1 illustrates the outer appearance of a dual-slot biological test meter according to the present invention.

FIG. 2 is a block diagram showing the control flow of the dual-slot biological test meter according to the present invention.

FIG. 3 is a circuit block diagram of the dual-slot biological test meter according to the present invention.

FIG. 4 illustrates the control waveform of the reference power supply according to the present invention.

FIG. 5 is a waveform chart showing a first test result reading method according to the present invention.

FIG. 6 is a waveform chart showing a second test result reading method according to the present invention.

### DETAILED DESCRIPTION OF THE INVENTION

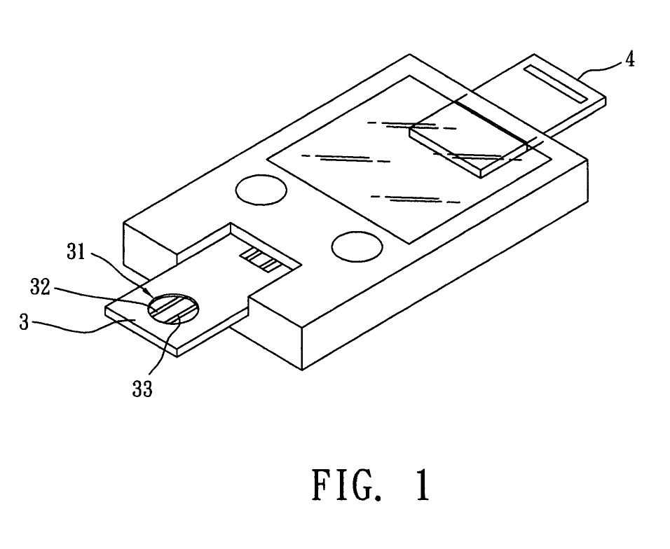

Referring to FIG. 1, a dual-slot biological test meter 1 is shown having two test slots 11,12 at two sides for receiving test strips 3 for examine a respective specific substance in the blood sample applied to each inserted test strip 3. A code card 4 may be inserted into one of the test slots 11,12 to set the parameters of the dual-slot biological test meter 1, defining one test slot 11,12 to examine one specific type of test strip 3 only.

The dual-slot biological test meter 1 can provide a respective reference voltage to the inserted test strip 3 subject to the type of the inserted test strip 3. For example, the dual-slot biological test meter 1 can provide one single power source, multiple power sources, saw wave, or different frequencies subject to the parameters provided by the code card 4, i.e., the dual-slot biological test meter 1 fits different commercially available biological test strips.

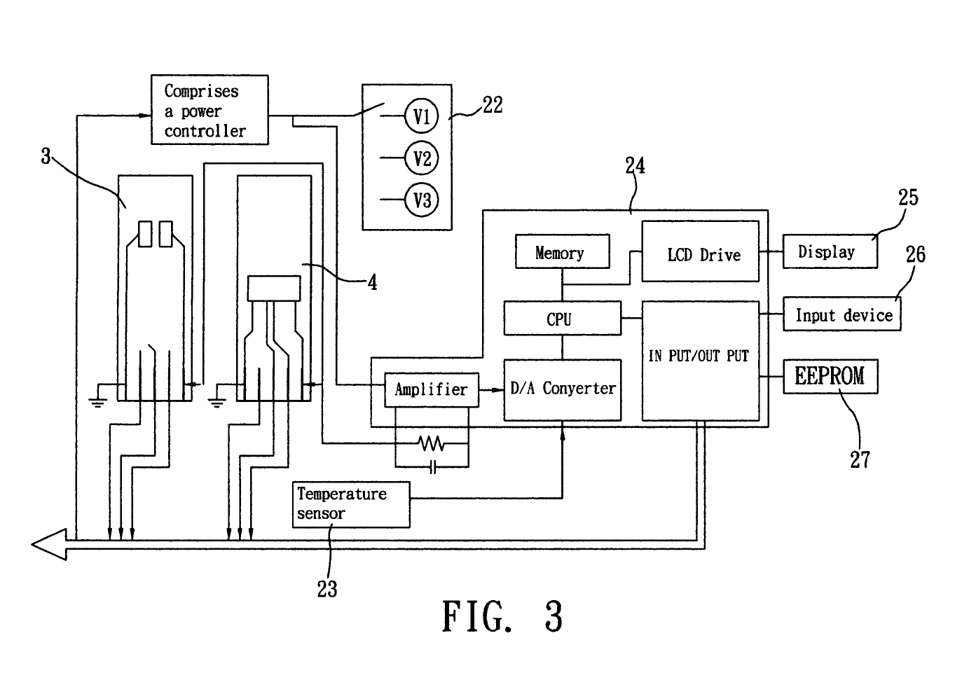

Referring to FIG. 3, the internal circuit 2 of the aforesaid dual-slot biological test meter 1 comprises a power controller 21, a reference voltage 22, a temperature sensor 23, a microprocessor 24, a display 25, an input device 26, and an EEPROM (electronically erasable programmable read only memory) 27. The display 25, the input device 26, and the EEPROM 27 are respectively connected to the microprocessor 24. Because the internal circuit 2 of the dual-slot biological test meter 1 is of the known art and not within the scope of the claims of the present invention, no further detailed description in this regard is necessary.

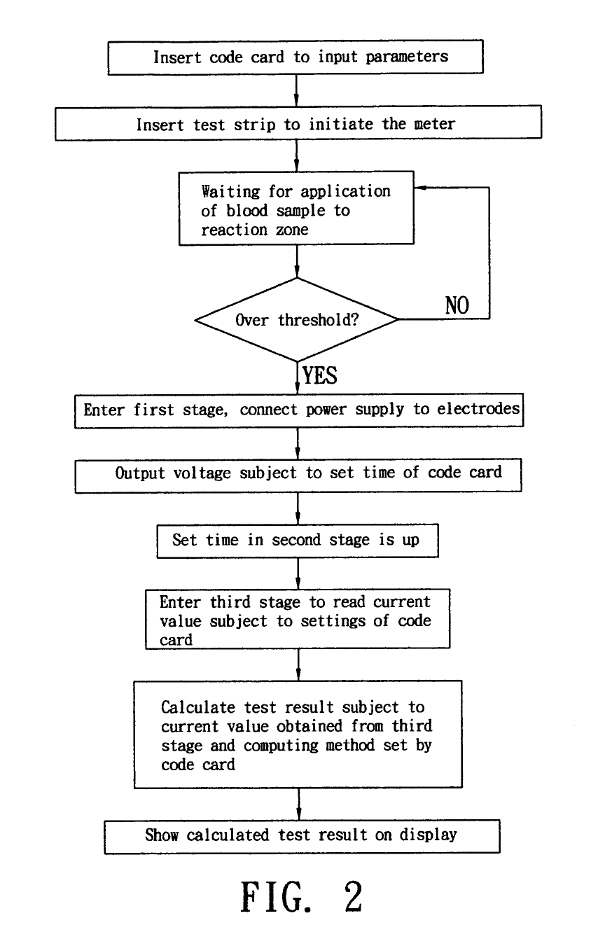

When in use, as shown in FIG. 2, the code card 4 is inserted into one test slot 12 of the dual-slot biological test meter 1 to input parameters into the dual-slot biological test meter 1, and then the assigned test strip 3 is inserted into the other test slot 11 to initiate the dual-slot biological test meter 1. After blood sample has been applied to the reaction zone 31 of the inserted test strip 3, the dual-slot biological test meter 1 judges if over the threshold or not. If not, return to stand-by status. If the result is over the threshold, the dual-slot biological test meter 1 enters the first stage to electrically connect the two electrodes 32,33 of the inserted test strip 3 and then enters the second stage to output a voltage to the electrodes 32,33 for a predetermined length of time set by the code card 4. When the set time is up, the dual-slot biological test meter 1 enters the third stage to read the current value subject to the setting of the code card 4, and then to calculate the test result subject to the computing method set by the code card 4, and then to show the test result on the display.

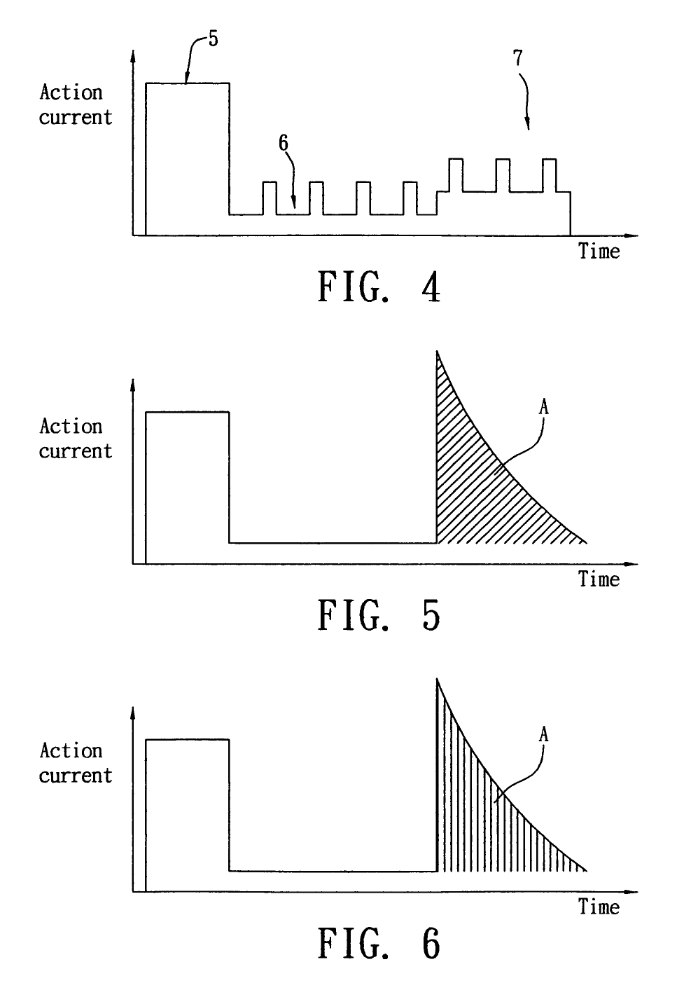

FIG. 4 illustrates the control waveform of the reference power supply. FIG. 5 illustrates the rest result waveform of the first method. FIG. 6 illustrates the test result waveform of the second method. As illustrated in FIG. 4, the control waveform of the reference power supply has three stages, namely, the first stage 5 to wait for receiving blood sample, the second stage 6 to waist for the reaction of the test strip 3, and the third stage 7 to read the result.

During the first stage 5, the two electrodes 32,33 of the test strip 3 are electrically connected (see FIG. 1). However, because the two electrodes 32,33 are in an open circuit status at this time, the current value measured by the dual-slot biological test meter 1 is below the threshold set in the code card 4. After dropping of blood sample to the reaction zone 31 of the test strip 3, the two electrodes 32,33 are closed; therefore the dual-slot biological test meter 1 enters the second stage 6.

When entered the second stage 6, a different reaction time is required for a different test item. For example, when examining blood glucose, the reaction time is about 4˜20 seconds; the reaction time is about 90˜180 seconds for cholesterol, or 30˜120 seconds for globubin. The predetermined reaction time for each test item is set in the code card 4 in factory subject to every commercially available type of test strip 3.

The third stage 7 begins immediately after the reaction time in the second stage 6 was up. The parameters for running the third stage 7 were set in the code card 4 in factory subject to every commercially available type of test strip 3. During this stage, the dual-slot biological test meter 1 provides a voltage ranging from 100 mV through 500 mV. The electricity connection time varies with the test items, for example, the reaction time is about 5˜20 seconds for glucose, 5˜30 seconds for cholesterol, or 5˜60 seconds for globubin. These time settings were set in the EEPROM of the code card 4 in factory subject to every commercially available type of test strip 3.

When applying voltage during the third stage 7, the value of electric current measured between the two electrodes 32,33 of the inserted test strip 3 is obtained after reaction of the reagent of the inserted test strip 3 with the test item blood sample.

The test result can be read by means of two methods. Referring to FIG. 5, the first method is to read the test result by means of the integration formula of:
A (area)=∫di/dt

For example, if the concentration of glucose in blood measured is 50 mg/dL, 100 mg/dL, 150 mg/dL, or 200 mg/dL, the integration value shall be 5000, 10000, 15000, or 20000 respectively. By means of linear regression, it is obtained that blood glucose concentration (mg/dL)=0.01× area A.

If the area obtained is 18000 when dropped one drop of blood sample of unknown concentration, the glucose concentration shall be 0.01× area A, i.e., 0.01×18000=180 mg/dL.

Referring to FIG. 6, the second method is to obtain the test result by means of reading the reading of the test sample at a predetermined time interval and then adding up the readings and then calculating the test result through a compensation or conversion procedure. For example, if the glucose concentration is 50 mg/dL at each reading when set to read current value per every 0.5 second within the reaction time of 5 seconds, the total number of readings will be 9 (at 0.5 second, 1.0 second, 1.5 seconds, 2.0 seconds, 2.5 seconds, 3.0 seconds, 3.5 seconds, 4.0 seconds, 4.5 seconds, 5.0 seconds) and the sum of the readings will be 500; if the concentration is 100, 150, 200 mg/dL respectively, the sum will be 1000, 1500, 2000 respectively, by means of linear regression, it is obtained that
blood glucose concentration (mg/dL)=0.01× area A.

If the area of 1800 is obtained after application of a blood sample of unknown concentration to the inserted test strip, the concentration of glucose of the blood sample will be blood glucose concentration (mg/dL)=0.01×1800 (area A)=180 mg/dL.

The invention further provides a calibration function. When inserted a calibrate card into one test slot of the dual-slot biological test meter, the dual-slot biological test meter automatically calibrate the linearity, preventing an examination error due to the aged problem of one component part.

The invention also provides the function of automatically examine the validity of the inserted test strip, preventing an examination error due to the use of an expired test strip.

Further, after insertion of a test strip into the dual-slot biological test meter, the dual-slot biological test meter automatically judges the type of the inserted test strip subject to the parameters inputted by the inserted test strip.

Although a particular embodiment of the invention has been described in detail for purposes of illustration, various modifications and enhancements may be made without departing from the spirit and scope of the invention. Accordingly, the invention is not to be limited except as by the appended claims.

What is claimed is:

1. A dual-slot biological test meter test procedure comprising the steps of:
a) inserting a code card into a first test slot of a dual-slot biological test meter to input predetermined parameters into said dual-slot biological test meter and then inserting a test strip into a second test slot of said dual-slot biological test meter to initiate said dual-slot biological test meter;
b) judging if the result is over the threshold or not after application of the blood sample to the reaction zone of the inserted test strip, and then entering a first state to electrically connect the two electrodes of the inserted test, strip if the result is over the threshold, and then entering a second stage to output a voltage to the electrodes of the inserted test strip for a predetermined length of time set by said code card;
c) entering a third stage to read the current value subject to the setting of said code card when said predetermined length of time is up, and then to calculate the test result through a computing method set by said code card subject to the current value obtained, and then to show the test result on a display.

2. The dual-slot biological test meter test procedure as claimed in claim 1, wherein said dual-slot biological test meter comprises a power controller, a reference voltage, a temperature sensor, a microprocessor, a display, an input device, and an EEPROM (electronically erasable programmable read only memory), said display and said input device and said EEPROM being respectively electrically connected to said microprocessor.

3. The dual-slot biological test meter test procedure as claimed in claim 2, wherein said reference voltage is within 100˜500 mV.

4. The dual-slot biological test meter test procedure as claimed in claim 1, wherein the reaction time for reaction of said test strip with the applied blood sample during said second state is about 4˜20 seconds for glucose, about 90˜180 seconds for cholesterol, about 30˜120 seconds for globubin.

5. The dual-slot biological test meter test procedure as claimed in claim 4, wherein the reaction time during said second stage is set in said code card in factory subject to each type of test strip.

6. The dual-slot biological test meter test procedure as claimed in claim 1, wherein the reaction time during said third stage is about 5˜20 seconds for glucose, about 5˜30 seconds for cholesterol, about 5˜60 seconds for globubin.

7. The dual-slot biological test meter test procedure as claimed in claim 6, wherein the reaction tine during said third stage is set in said code card in factory subject to each type of test strip.

8. The dual-slot biological test meter test procedure as claimed in claim 1, wherein the test result is read by means of the integration formula of:
A (area)=∫di/dt; concentration (mg/dL)=0.01× area (A).

9. The dual-slot biological test meter test procedure as claimed in claim 1, wherein the test result is read by means of reading the reading of the test sample at a predetermined time interval and then adding up the readings and then calculating the test result through a compensation or conversion procedure.

10. The dual-slot biological test meter test procedure as claimed in claim 1, further comprising the step of inserting a calibrate card into one of the two test slots of said dual-slot biological test meter to examine the linearity of said dual-slot biological test meter.

11. The dual-slot biological test meter test procedure as claimed in claim 1, wherein said dual-slot biological test meter has the function of automatically examining the validity of the inserted test strip.

12. The dual-slot biological test meter test procedure as claimed in claim 1, further comprising the step of automatically judging the type of the inserted test strip subject to the parameters inputted by the inserted test strip into said dual-slot biological test meter upon insertion of the test strip into said dual-slot biological test meter.
